(19) United States
(12) Patent Application Publication
(10) Pub. No.: US 2025/0235410 A1
(43) Pub. Date: Jul. 24, 2025

(54) WATER-SOLUBLE CANNABIDIOL MICROCAPSULES AND PREPARATION METHOD THEREOF

(71) Applicant: Northeast Agricultural University, Harbin City (CN)

(72) Inventors: Zhanmei JIANG, Harbin City (CN); Juncai HOU, Harbin City (CN); Rui YU, Harbin City (CN); Jiage MA, Harbin City (CN); Hongyu LI, Harbin City (CN)

(21) Appl. No.: 18/413,182

(22) PCT Filed: Jun. 12, 2023
(86) PCT No.: PCT/CN2023/099652

§ 371 Date: Feb. 21, 2025

(30) Foreign Application Priority Data
Jun. 30, 2022 (CN) 202210778496.X

(51) Int. Cl. A61K 9/50; A61K 31/05

(52) U.S. Cl. CPC A61K 9/5052; A61K 9/5089; A61K 31/05

(57) ABSTRACT

A preparation method of water-soluble cannabidiol microcapsules and an application thereof are provided, belong to the field of biotechnology. The preparation method includes: adding galactose into an aqueous solution of whey protein isolate, adjusting a pH value, reacting, and cooling to obtain Maillard Reaction Products (MRPs) solution of whey protein isolate-galactose; adding an emulsifier into the MRPs solution, adjusting a pH value, high-speed shearing and stirring in the oil solution with cannabidiol (CBD) to obtain crude emulsion, and homogenizing the crude emulsion to obtain a Maillard Reaction Products-cannabidiol (MRPs-CBD) emulsion; and pre-freezing and vacuum freeze-drying the MRPs-CBD emulsion in turn to obtain the water-soluble cannabidiol microcapsules.

### TECHNICAL FIELD

The application relates to the field of biotechnology, and in particular to water-soluble cannabidiol microcapsules and a preparation method thereof.

### BACKGROUND

Cannabidiol (CBD) is a non-addictive component in cannabis extract, which has many physiological activities such as antioxidant, anticancer and anti-inflammatory. Due to the low water solubility and poor stability, CBD is easily influenced by external environment (temperature, light and oxygen), processing and storage conditions and digestive tract environment (pH value, enzymes and other substances), which leads to many restrictions in the application of CBD. Therefore, it is of great significance for the application of CBD to provide a preparation method of water-soluble cannabidiol microcapsules, which increases the water solubility of CBD, improves the stability of CBD in adverse environment and improves the slow-release ability of CBD.

### SUMMARY

Based on the above contents, the application provides water-soluble cannabidiol microcapsules and a preparation method thereof, which effectively improves the water solubility and storage stability of the cannabidiol microcapsules and enhances the practicability of cannabidiol by utilizing the good emulsification and oxidation resistance of glycosylated products.

In order to achieve the above objectives, the present application provides the following schemes.

One of the technical schemes of the application is a preparation method of water-soluble cannabidiol microcapsules, which includes the following steps:
step 1, adding galactose into an aqueous solution of whey protein isolate, adjusting a pH value to be alkaline, reacting, and cooling to obtain a glycosylation product solution of whey protein isolate-galactose;
step 2, adding an emulsifier into the glycosylation product solution of whey protein isolate-galactose, adjusting a pH value to be acidic or neutral, mixing and stirring with the an oil solution of the cannabidiol to obtain a crude emulsion, and homogenizing the crude emulsion to obtain a Maillard Reaction Products-cannabidiol (MRPs-CBD) emulsion; and
step 3, pre-freezing and vacuum freeze-drying the MRPs-CBD emulsion in turn to obtain the glycosylation product-based water-soluble cannabidiol microcapsules.

Optionally, in the step 1, a concentration of the aqueous solution of whey protein isolate is 1-6 weight percent (wt %); adjusting a pH value is specifically adjusting pH=8-10; the reacting is specifically reacting at 70-90° C. for 1-5 hours (h).

Optionally, in the step 2, a mass ratio of whey protein to the galactose in the glycosylation product solution of whey protein isolate-galactose is 1:1; the emulsifier is one of tea saponins, Steviol glycosides, Gynostemma pentaphyllum saponins or Tribulus terrestris saponins; the emulsifier accounts for 0.05-5%, preferably 0.2-0.4%, more preferably 0.35% of a mass of glycosylation product solution of whey protein isolate-galactose.

Optionally, in the step 2, a concentration of the oil solution of cannabidiol is 2-12 wt %, preferably 8-12 wt %, more preferably 11.43%; an oil in the oil solution of cannabidiol is vegetable oil; the vegetable oil is one of sunflower seed oil, soybean oil, olive oil, peanut oil or corn oil.

Optionally, in the step 2, a volume ratio of the glycosylation product solution of whey protein isolate-galactose to the oil solution of cannabidiol is 6:4-9:1, preferably 6:4-8:2, and more preferably 6:4.

Optionally, in the step 2, adjusting a pH value is specifically adjusting pH=6-7, preferably pH=6.7-6.9, more preferably pH=6.85; the stirring is specifically emulsifying at 6000-10000 revolutions per minute (r/min) for 2-5 minutes (min); the homogenizing is specifically 4-6 times of cycles at 50-80 megapascals (MPa).

Optionally, in the step 3, the pre-freezing is specifically pre-freezing with liquid nitrogen for 1 min.

Optionally, in the step 3, the vacuum freeze-drying is specifically freeze-drying for 18-30 h at −50 to −70° C. and 2-10 MPa.

According to another technical scheme of the application, water-soluble cannabidiol microcapsules are prepared by the preparation method mentioned above.

Another technical scheme of the application is an application of the water-soluble cannabidiol microcapsules in preparing anti-oxidation, anti-cancer and anti-inflammatory drugs.

The application discloses the following technical effects.

The wall material of the water-soluble cannabidiol microcapsules prepared by the application adopts whey protein glycosylation product, the core material adopts vegetable oil dissolving CBD, the emulsifier adopts natural emulsifier, the microencapsulation method adopts vacuum freeze-drying method, and pre-freezing adopts liquid nitrogen for quick freezing, so that the damage to the microcapsule structure caused by water crystallization during pre-freezing is effectively reduced. Meanwhile, the preparation conditions of the glycosylation product-based water-soluble cannabidiol microcapsules are optimized through a response surface model, and the encapsulation efficiency of the obtained microcapsules is (76.02±0.06) %, the loading capacity is (27.82±0.27) %, and the solubility of CBD in ultrapure water is (276.33±1.34) mg/mL at normal temperature and normal pressure, which increases the water solubility of CBD to facilitate its application in production, improves its stability in adverse environment to prolong its shelf life, and improves its slow-release ability to enhance targeted drug administration and further improves bioavailability of CBD. It is observed by scanning electron microscope that microcapsules are irregular particles with compact structure, and have pores and depressions on the surface. The radical scavenging rate of microcapsules in this application is significantly higher than that of unencapsulated CBD (P<0.05), and the microcapsules have strong antioxidant activity.

### BRIEF DESCRIPTION OF THE DRAWINGS

In order to explain the embodiments of the present application or the technical schemes in the prior art more clearly, the drawings needed in the embodiments will be briefly introduced below. Obviously, the drawings described below are only some embodiments of the present application, and other drawings may be obtained according to these drawings without creative efforts for ordinary people in the field.

FIG. 1 shows a design and results of a response surface experiment of an embodiment of the present application.

FIG. 2 shows optimal preparation conditions and results of the embodiment of the present application.

FIG. 3A is a microstructure diagram of unencapsulated cannabidiol (CBD) under scanning electron microscope with magnification of 1,200 times in the embodiment of the present application.

FIG. 3B is a microstructure diagram of the unencapsulated CBD under scanning electron microscope with magnification of 5,000 times in the embodiment of the present application.

FIG. 4A is a microstructure diagram of Maillard Reaction Products-cannabidiol (MRPs-CBD) microcapsules under scanning electron microscope with magnification of 1,200 times in the embodiment of the present application.

FIG. 4B is a microstructure diagram of the MRPs-CBD microcapsules under scanning electron microscope with magnification of 5,000 times in the embodiment of the present application.

FIG. 5A is results of 1,1-Diphenyl-2-picrylhydrazyl (DPPH) radical scavenging rate of the MRPs-CBD microcapsules according to the embodiment of the present application.

FIG. 5B is results of 2,2′-azino-bis (3-ethylbenzothiazoline-6-sulfonic acid) (ABTS⁺) radical cation scavenging rate of the MRPs-CBD microcapsules in the embodiment of the present application.

FIG. 6 shows an influence of temperature on stability of the MRPs-CBD microcapsules in the embodiment of the present application.

FIG. 7 shows an influence of environmental pH value on the stability of MRPs-CBD microcapsules according to the embodiment of the present application.

FIG. 8A shows an influence of oxygen on the stability of MRPs-CBD microcapsules at 4° C. in the embodiment of the present application.

FIG. 8B shows an influence of oxygen on the stability of MRPs-CBD microcapsules at 25° C. in the embodiment of the present application.

FIG. 9 shows an influence of incandescent light on the stability of MRPs-CBD microcapsules in the embodiment of the present application.

FIG. 10 shows an influence of ultraviolet irradiation on the stability of MRPs-CBD microcapsules in the embodiment of the present application.

FIG. 11 shows release results of the MRPs-CBD microcapsules in simulated gastric juice in the embodiment of the present application.

FIG. 12 shows the release results of MRPs-CBD microcapsules in simulated intestinal fluid in the embodiment of the present application.

FIG. 13 shows the in-turn release results of the MRPs-CBD microcapsules in the simulated gastric juice and the intestinal fluid in the embodiment of the present application.

FIG. 14 shows an influence of oil phase types on an encapsulation efficiency of the MRPs-CBD microcapsules.

FIG. 15 shows the influence of oil phase types on solubility of CBD in the MRPs-CBD microcapsules.

FIG. 16 shows an influence of oil phase content on the encapsulation efficiency of MRPs-CBD microcapsules in the embodiment of the present application.

FIG. 17 shows the influence of oil phase content on the solubility of CBD in MRPs-CBD microcapsules in the embodiment of the present application.

FIG. 18 shows an influence of CBD content on the encapsulation efficiency of MRPs-CBD microcapsules.

FIG. 19 shows the influence of CBD content on the solubility of CBD in MRPs-CBD microcapsules in the embodiment of the present application.

FIG. 20 shows an influence of tea saponin content on the encapsulation efficiency of MRPs-CBD microcapsules in the embodiment of the present application.

FIG. 21 shows the influence of tea saponin content on the solubility of CBD in MRPs-CBD microcapsules in the embodiment of the present application.

FIG. 22 shows an influence of pH value of water phase on the encapsulation efficiency of MRPs-CBD microcapsules.

FIG. 23 shows the influence of pH value of water phase on the solubility of CBD in MRPs-CBD microcapsules.

### DETAILED DESCRIPTION OF THE EMBODIMENTS

A number of exemplary embodiments of the present application will now be described in detail, and this detailed description should not be considered as a limitation of the present application, but should be understood as a more detailed description of certain aspects, characteristics and embodiments of the present application.

It should be understood that the terminology described in the present application is only for describing specific embodiments and is not used to limit the present application. In addition, for the numerical range in the present application, it should be understood that each intermediate value between the upper limit and the lower limit of the range is also specifically disclosed. The intermediate value within any stated value or stated range and every smaller range between any other stated value or intermediate value within the stated range are also included in the present application. The upper and lower limits of these smaller ranges may be independently included or excluded from the range.

Unless otherwise specified, all technical and scientific terms used herein have the same meaning as commonly understood by one of ordinary skill in the art to which this application relates. Although the present application only describes the preferred methods and materials, any methods and materials similar or equivalent to those described herein may also be used in the practice or testing in the present application. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present application without departing from the scope or spirit of the present application. Other embodiments will be apparent to the skilled person from the description of the application. The description and embodiments of that present application are exemplary only.

The terms “comprising”, “including”, “having” and “containing” used in this article are all open terms, which means including but not limited to.

Unless otherwise specified, “room temperature” in the present application refers to 15-30° C.

### Embodiment 1

(1) Preparation of external water phase: Whey Protein Isolate (WPI) powder is dissolved in distilled water to prepare a solution with a concentration of 4% (weight percent, wt %), and the WPI powder is stirred at room temperature for 2 h for full dissolution. Galactose with the same mass as WPI powder is added into WPI solution to prepare mixed solution; the pH value of the mixed solution is adjusted as 8.0, the mixed solution is put in a closed container and subjected to water bath reaction at 90° C. for 4 h. After the reaction, the mixed solution is taken out and quickly cooled in ice bath to obtain the glycosylation product solution of whey protein isolate-galactose, labeled as Maillard Reaction Products (MRPs), and stored at 4° C. for later use.
(2) Preparation of oil phase: a certain amount of cannabidiol is respectively weighed and dissolved in sunflower seed oil, so that the content of cannabidiol (CBD) is 2%, 4%, 6%, 8%, 10% and 12% (wt %), and CBD is completely dissolved by stirring at room temperature.
(3) Preparation of emulsion: the stored external water phase is taken out, followed by adding tea saponin as well as adjusting pH value according to Table 1, and oil solutions with different cannabidiol contents are added, the mixed solutions are emulsified respectively in a high-speed disperser at 10000 r/min for 4 min to prepare crude emulsions, then the crude emulsions are homogenized with an ultra-high pressure homogenizer, and homogenized for 6 times at 80 MPa to form cannabidiol-carried oil/water (O/W) emulsions of the glycosylation product of whey protein, which are marked as MRPs-CBD emulsions.

TABLE 1
Factors and levels in single factor experiment of
preparation conditions of MRPs-CBD microcapsules

| Factor | Level |
|---|---|
| Oil phase content (v %) | 10, 20, 30, 40 |
| CBD content (w %) | 0, 2, 4, 6, 8, 10, 12 |
| Tea saponin content | 0, 0.05, 0.1, 0.2, 0.3, 0.4, 0.5 |
| (w %) | |
| pH value of external | 6.6, 6.7, 6.8, 6.9, 7.0 |
| water phase | |

(4) Preparation of MRPs-CBD microcapsules: the prepared MRPS-CBD emulsions are put in a flat plate, weighed, wrapped with tin foil, put in liquid nitrogen for 1 min, pre-frozen into solids, vacuum freeze-dried at −60° C. and 10 MPa for 24 h to make powder, the freeze-dried powder is taken out, weighed again, and transferred to a brown centrifuge tube for freezing and storage at −20° C. The powder is the cannabidiol-carried microcapsules (the glycosylation product-based water-soluble cannabidiol microcapsules) of glycosylation product of whey protein, which are labeled as MRPs-CBD microcapsules, microcapsules for short, and the whole process is carried out in the dark to reduce the oxidation of CBD.
(5) The standard curve of CBD is plotted by high performance liquid chromatography and the CBD content in MRPs-CBD microcapsules prepared under different conditions is measured, and then the encapsulation efficiency, loading capacity and solubility of CBD in microcapsules are calculated based on the standard curve and the measured CBD content.

Plotting the standard curve of CBD: 0.1 gram (g) of cannabidiol standard is accurately weighed and dissolved in acetonitrile, and put in a 100 milliliter (mL) brown volumetric flask to prepare 1000 microgram per milliliter (μg/mL) cannabidiol mother liquor. The standard solutions with CBD concentrations of 10 μg/mL, 20 μg/mL, 40 μg/mL, 60 μg/mL and 80 μg/mL are prepared respectively through diluting by acetonitrile and stored at −10° C. The high performance liquid chromatography is used for detection, and the chromatographic conditions are as follows: C18 chromatographic column (4.6×100 mm, 5 μm, Agilent), acetonitrile solution with mobile phase of 30%, isocratic elution for 10 min, chromatographic column equilibrium and elution flow rate of 1 mL/min, column temperature of 37° C., injection volume of 10 μL, detection wavelength of 220 nm. A sample injection analysis is made under the above conditions. The standard curve is plotted with CBD concentration as abscissa and peak area as ordinate, and the standard curve of CBD is calculated as y=36.09x−21.73, R²>0.9999.

Determination of CBD content in microcapsules: 0.1 g of sample is weighed, added with 3 mL ultrapure water and stirred evenly, eddied until the sample is completely dissolved, then added with 5 mL acetonitrile and eddied for 30 seconds(s), and sonicated for 30 min in an ultrasonic cleaner at 37° C. and 20 watts (W). The sonicated sample is centrifuged at 5000 r/min for 3 min. After centrifugation, the supernatant is taken, subjected to a 220 μm organic filter membrane, and put into a brown bottle, and a peak area of the sonicated sample is measured at the wavelength of 220 nm, and then substituted into the standard curve, so as to calculate the CBD content in MRPs-CBD microcapsules.

Determination of encapsulation efficiency and loading capacity of CBD in microcapsules: the total CBD content in microcapsules is measured according to the above method, the total CBD input mass and microcapsule mass are measured and recorded when microcapsules are prepared, and the encapsulation efficiency and loading capacity of microcapsules are calculated according to the following formula:

$$encapsulation efficiency of microcapsules ( \% ) = \frac{total CBD content in microcapsules}{total CBD input mass} \times 100$$

$$loading capacity of microcapsules ( \% ) = \frac{total CBD mass in microcapsules}{microcapsule mass} \times 100$$

Determination of the solubility of CBD in microcapsules: microcapsules are dissolved in 1 mL of water at room temperature to obtain the maximum dissolved mass M, and the solubility of CBD in microcapsules is calculated according to the following formula:

$$solubility of CBD in microcapsules ( \frac{mg}{mL} ) = M \times loading capacity \times 1000$$

(6) Influence of oil phase types on MRPs-CBD microcapsules

Corn oil, sunflower seed oil, soybean oil, olive oil and peanut oil of vegetable oil are selected as oil phases, the content of oil phase is fixed as 40%, the content of external water phase is fixed as 60%, the content of CBD is fixed as 2%, the content of tea saponins is fixed as 0.05%, and the pH value of external water phase is adjusted as 6.6 to prepare MRPs-CBD emulsions and its microcapsules, and the influence of oil phase types on MRPs-CBD emulsions and its microcapsules are explored.

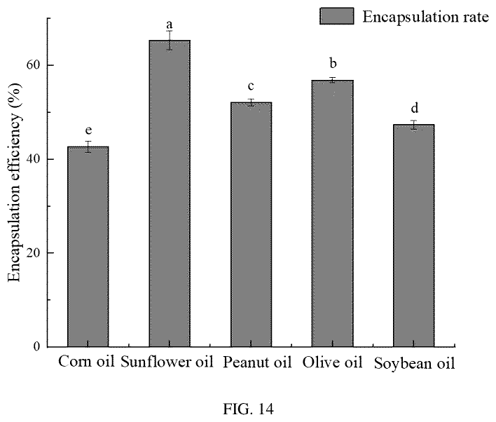

FIG. 14 shows the influence of oil phase types on the encapsulation efficiency of MRPs-CBD microcapsules. As can be seen from FIG. 14, the encapsulation efficiencies of MRPs-CBD microcapsules prepared from the corn oil, the sunflower seed oil, the soybean oil, the peanut oil and the olive oil are (42.65±1.20) %, (65.33±2.02) %, (52.13±0.77) %, (56.89±0.57) % and (47.36±0.89) % respectively.

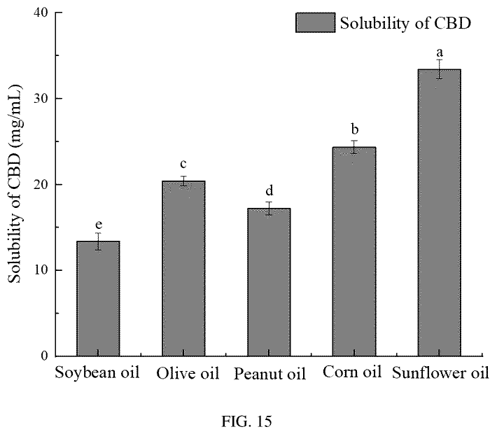

FIG. 15 shows the influence of oil phase types on the solubility of CBD in MRPs-CBD microcapsules. It can be seen from FIG. 15 that the solubility of CBD in MRPs-CBD microcapsules prepared from the corn oil, the sunflower seed oil, the soybean oil, the peanut oil and the olive oil is (13.37±0.98) mg/mL, (20.42±0.54) mg/mL and (17.22±0.76) mg/mL, (24.39±0.77) mg/mL, and (33.43±1.102) mg/mL respectively.
(7) Influence of oil phase content on MRPs-CBD microcapsules

The sunflower seed oil is selected as oil phase, the content of oil phase and external water phase is adjusted to make the oil phase content 10%, 20%, 30% and 40%, the external water phase content 90%, 80%, 70% and 60% respectively, the CBD content is fixed as 2%, the tea saponin content as 0.05%, and the pH value of external water phase is adjusted as 6.6 to prepare MRPs-CBD emulsions and its microcapsules, and the influence of oil phase content on MRPs-CBD emulsions and its microcapsules are explored.

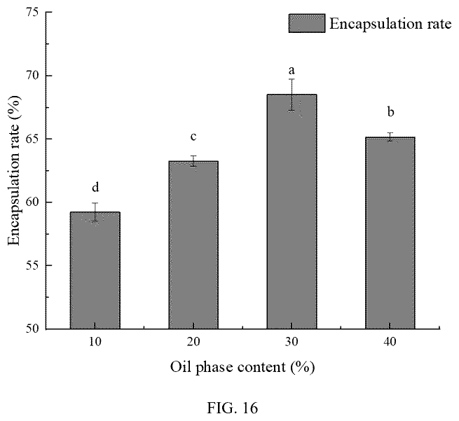

FIG. 16 shows the influence of oil phase content on the encapsulation efficiencies of MRPs-CBD microcapsules. As can be seen from FIG. 16, when the proportion of oil phase is 30%, the encapsulation efficiency reaches the highest (68.52±1.23) %, and when the proportion of the oil phase is further increased to 40%, the encapsulation efficiency decreases to (65.17±0.32) %, which is 5.94% higher than that of microcapsules with the proportion of oil phase of 10%.

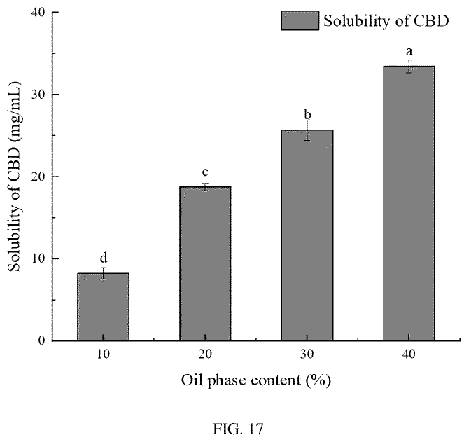

FIG. 17 shows the influence of oil phase content on the solubility of CBD in MRPs-CBD microcapsules. As can be seen from FIG. 17, the solubility of CBD in MRPs-CBD microcapsules with oil phase content of 40% is (33.45±0.81) mg/mL, which is three times higher than that of MRPs-CBD microcapsules with oil phase content of 10%, and the difference is extremely significant.
(8) Influence of CBD content on MRPs-CBD microcapsules

The sunflower seed oil is selected as oil phase, CBD with different mass is dissolved in the sunflower seed oil at room temperature, and the oil phase content is fixed as 40%, tea saponin content as 0.05%, and the pH value of external water phase is adjusted as 6.6 to prepare MRPs-CBD emulsions and its microcapsules, so that the CBD content (w %) in the emulsions is 0%, 2%, 4%, 6%, 8% and 10 respectively for studying the influence of CBD content on MRPs-CBD emulsions and its microcapsules.

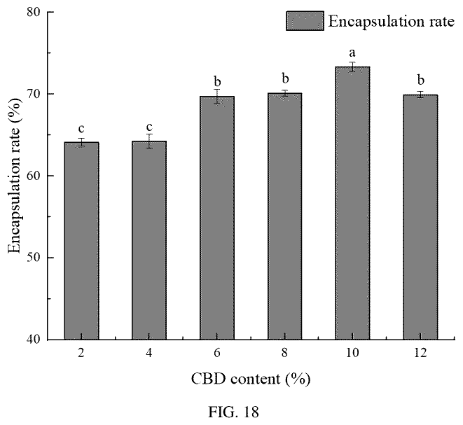

FIG. 18 shows the influence of CBD content on the encapsulation efficiencies of MRPs-CBD microcapsules. As can be seen from FIG. 18, when the CBD content increases from 2% to 12%, the encapsulation efficiencies of MRPs-CBD microcapsules first increase and then decrease. When the CBD content is 10%, the encapsulation efficiency reaches the peak, which is (73.34±0.56) %.

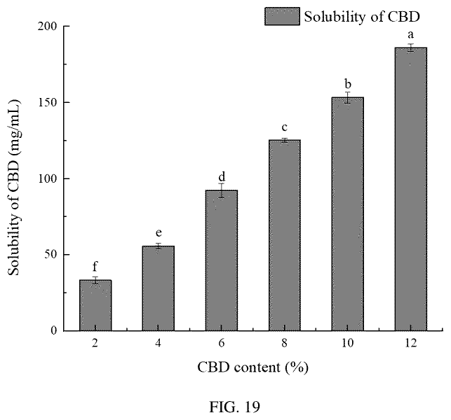

FIG. 19 shows the influence of CBD content on the solubility of CBD in MRPs-CBD microcapsules. As can be seen from FIG. 19, when the content of CBD is 2%, 4%, 6%, 8%, 10% and 12%, the solubility of CBD in the prepared MRPs-CBD microcapsules is (33.34±2.32) mg/mL, (55.83±1.73) mg/mL, (92.37±4.72) mg/mL, (125.38±1.38) mg/mL, (153.43±3.67) mg/mL, and (186.23±2.44) mg/mL respectively.
(9) Influence of tea saponin content on MRPs-CBD microcapsules

The sunflower seed oil is selected as the oil phase, the oil phase content is fixed as 40% and CBD content as 2%, the tea saponins with different mass is dissolved in the external water phase, and the pH value of the external water phase is adjusted as 6.6, so that the tea saponin content (w %) in MRPs-CBD emulsions is 0%, 0.05%, 0.1%, 0.2%, 0.3%, 0.4%, respectively for preparing MRPs-CBD emulsions and its microcapsules and studying the influence of tea saponin content on MRPs-CBD emulsions and its microcapsules.

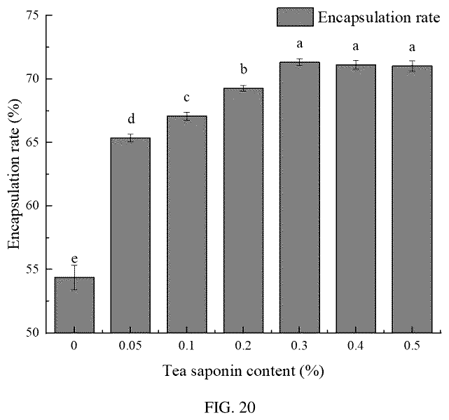

FIG. 20 shows the influence of tea saponin content on the encapsulation efficiencies of MRPs-CBD microcapsules. As can be seen from FIG. 20, when tea saponin content is 0.3%, the encapsulation efficiency of MRPs-CBD microcapsules is the largest, which is (71.32±0.26) %.

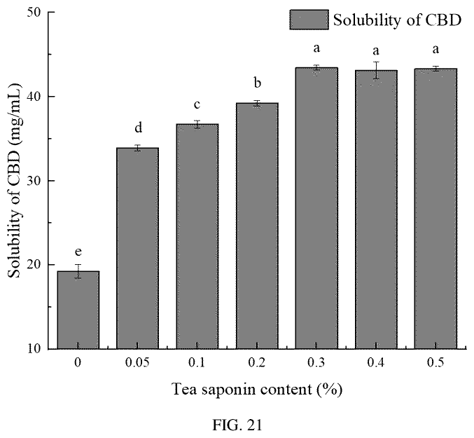

FIG. 21 shows the influence of tea saponin content on the solubility of CBD in MRPs-CBD microcapsules. It can be seen from FIG. 21 that when the tea saponin content is 0.3%, the solubility of CBD in MRPs-CBD microcapsules is the largest, which is (43.45±0.31) mg/mL, and the solubility of CBD is doubled compared with the blank group without tea saponins.
(10) Influence of pH value of external water phase on MRPs-CBD microcapsules

Sunflower seed oil is selected as the oil phase, the oil phase content is fixed as 40%, CBD content as 2% and tea saponin content as 0.05%, and the pH value of external water phase is adjusted as 6.6, 6.7, 6.8, 6.9 and 7.0 to prepare MRPs-CBD emulsions and its microcapsules and studying the influence of tea saponin content on MRPs-CBD emulsions and its microcapsules.

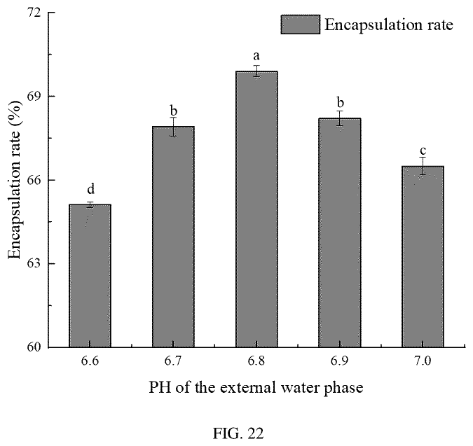

FIG. 22 shows the influence of pH value of external water phase on the encapsulation efficiencies of MRPs-CBD microcapsules. As can be seen from FIG. 22, when the pH value of the external water phase is 6.8, the encapsulation efficiency reaches a peak value of (69.91±0.20) %.

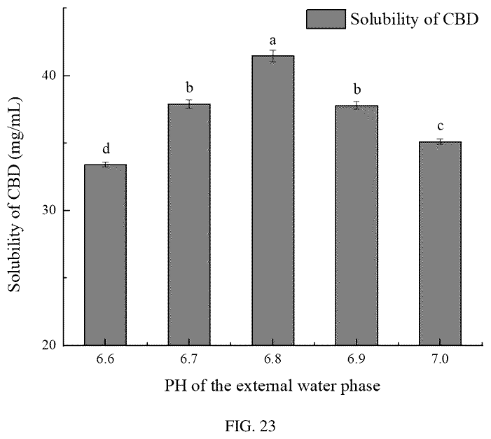

FIG. 23 shows the influence of pH value of external water phase on the solubility of CBD in MRPs-CBD microcapsules. It can be seen from FIG. 23 that the solubility of CBD in MRPs-CBD microcapsules reaches the peak value of (41.48±0.46) mg/mL when the pH value of the external water phase is 6.8. When the pH of the external water phase is increased to 7.0, the solubility of CBD in the microcapsules in water decreases.
(11) After obtaining the encapsulation efficiencies and solubility under each condition, the optimal four factors and corresponding three levels are determined as follows: oil phase content of 20%, 30% and 40%, CBD content of 8%, 10% and 12%, tea saponin content of 0.2%, 0.3% and 0.4%, and pH value of external water phase of 6.7, 6.8 and 6.9.

TABLE 2
Level coding table of response surface experiment
factors of MRPs-CBD microcapsules

| | | | | pH value of the |
|---|---|---|---|---|
| | Oil phase content | CBD content | Tea saponin content | external water |
| Coding | (A)/v % | (B)/w % | (C)/w % | phase (D) |
| −1 | 20 | 8 | 0.2 | 6.7 |
| 0 | 30 | 10 | 0.3 | 6.8 |
| 1 | 40 | 12 | 0.4 | 6.9 |

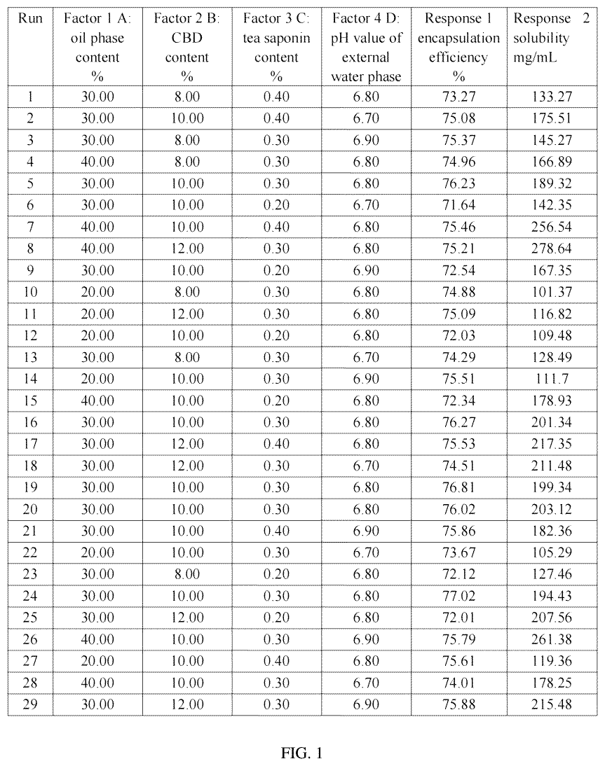

Response surface model Design-Expert analysis software and Box-Behnken are used to design experiment. Experiment designs and results of 19 groups of response surfaces are obtained with the encapsulation efficiency and CBD solubility of MRPs-CBD microcapsules as response values. An experiment is conducted for each group according to 19 groups of experiment designs, and the result values of each group are input (see FIG. 1). According to the software, the next operation is carried out to obtain the regression fitting equation.

$$The encapsulation efficiency ( \% ) = 76.47 + 0.082 A + 0.28 B + 1.51 C + 0.65 D + 0.01 AB - 0.12 AC - 0.015 AD + 0.59 BC + 0.072 BD - 0.03 CD - 0.69 {A}^{2} - 0.87 {B}^{2} - 2.08 {C}^{2} - 0.74 {D}^{2} .$$

$$Solubility of CBD ( mg / mL ) = 197.51 + 54.71 A + 37.05 B + 12.61 C + 11.85 D + 24.07 AB + 16.93 AC + 19.16 AD + 0.99 BC - 3.19 BD - 4.54 CD - 18.94 {A}^{2} - 10.77 {B}^{2} - 14.84 {C}^{2} - 13.91 {D}^{2} .$$

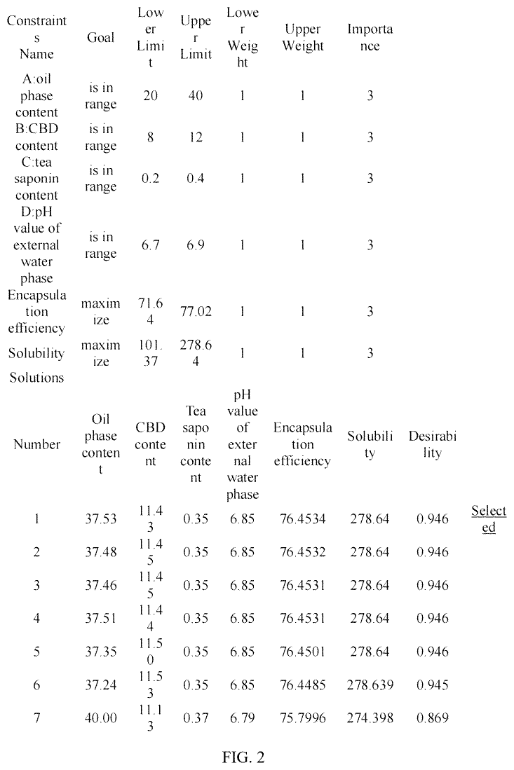

The operation is continued to get the optimal preparation conditions and prediction results (see FIG. 2).

The optimal preparation conditions are determined by using Design-Expert analysis software and combined solution method: oil phase content is 37.53%, CBD content is 11.43%, tea saponin content is 0.35%, and pH value of external water phase is 6.85. Under these conditions, the theoretical encapsulation efficiency is 76.46% and the theoretical solubility of CBD is 278.64 mg/mL. For the convenience of production, the preparation conditions are optimized as follows: oil phase content of 40%, CBD content of 12%, tea saponin content of 0.35% and pH value of external water phase of 6.85. After three parallel experiments, the encapsulation efficiency and solubility of CBD of microcapsules are (76.02±0.06) % and (278.23±0.27) mg/mL, respectively. There is a high degree of agreement between the experiment values of response values and the predicted values of regression equation, which shows that the model is effective. The microcapsules prepared under this preparation conditions are selected for subsequent tests.

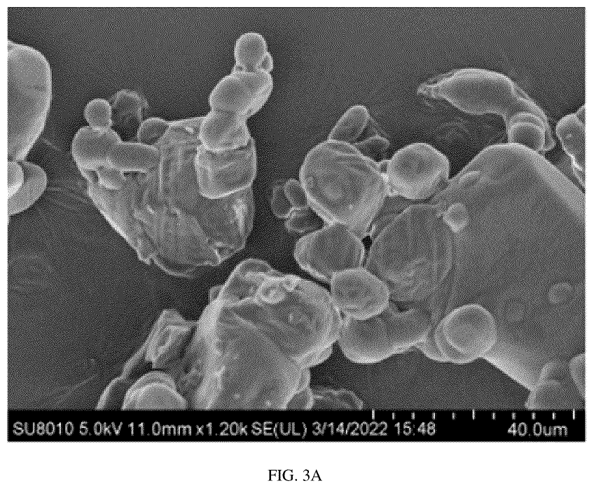

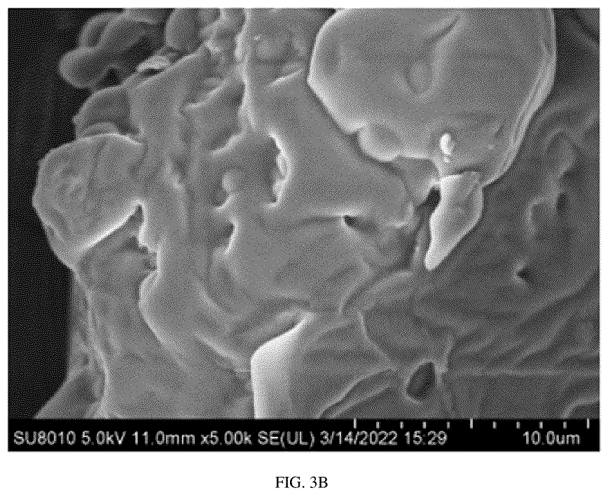

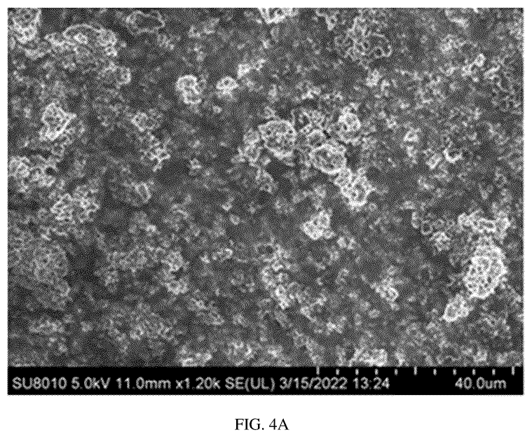

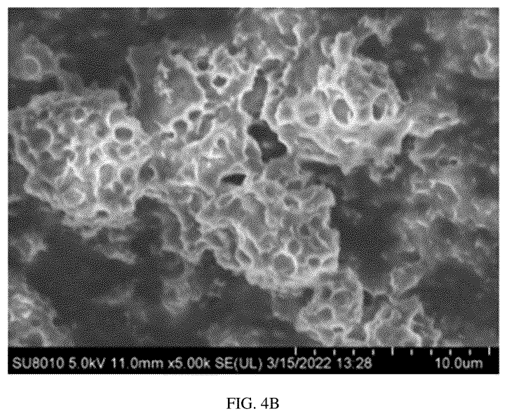

The unencapsulated CBD powder and MRPs-CBD microcapsules with encapsulation efficiency and solubility of CBD of (76.02±0.06) % and (278.23±0.27) mg/mL respectively are observed under a scanning electron microscope to obtain FIG. 3A-FIG. 3B (FIG. 3A is a microstructure diagram of unencapsulated CBD under scanning electron microscope with magnification of 1,200 times; FIG. 3B is a microstructure diagram of unencapsulated CBD under scanning electron microscope with magnification of 5,000 times), and FIG. 4A-FIG. 4B (FIG. 4A is a microstructure diagram of Maillard Reaction Products-cannabidiol (MRPs-CBD) microcapsules under scanning electron microscope with magnification of 1,200 times; FIG. 4B is a microstructure diagram of the MRPs-CBD microcapsules under scanning electron microscope with magnification of 5,000 times).

It can be seen from FIG. 3A, and FIG. 3B that the unencapsulated CBD powder is an irregular crystal with uneven particle size and unsmooth surface, and the particle diameter exceeds 400 μm. It can be seen from FIG. 4A and FIG. 4B that MRPs-CBD microcapsules are irregular particles, with rough surface and porous structure on the surface. The porous structure may be micropores left by the sublimation of ice crystals formed by condensation of water in pre-frozen emulsion during drying, and the inside is a tight mesh structure, which is capable of enhancing the slow release effect of microcapsules on CBD.

The antioxidant activity of glycosylation product-based water-soluble cannabidiol microcapsules is analyzed through radical scavenging rate. The scavenging ability of 1,1-Diphenyl-2-picrylhydrazyl (DPPH) and, 2′-azino-bis (3-ethylbenzothiazoline-6-sulfonic acid (ABTS⁺) radical cation is mainly studied.

Determination of DPPH radical scavenging rate: 1 mL of CBD with different concentrations, methanol solution of microcapsules with different concentrations and 4 mL of 0.1 mmol/L DPPH solution (DPPH dissolved in 95% ethanol) are mixed evenly, and kept in the dark for 30 min at room temperature. Using 95% ethanol as reference solution, the absorbance at 517 nm is read. The absorbance value of the sample mixed with DPPH ethanol solution is recorded as A, the absorbance value of the sample mixed with 95% ethanol solution is Ai, and the absorbance value of the single DPPH ethanol solution is Aj. The calculation formula is as follows:

$$DPPH radical scavenging rate ( \% ) = ( 1 - \frac{A - Ai}{Aj} ) \times 100$$

Determination of ABTS⁺ radical scavenging rate: 10 mg of ABTS and 2.9 mg of potassium persulfate are taken, dissolved in 10 mL of 0.01 mol/L sodium phosphate buffer, and stored in the dark at 25° C. for 15 h for later use. 2.5 mL ABTS solution is taken, added with 20 mL of 0.01 mol/L sodium phosphate buffer, and diluted with distilled water so that the absorbance at 734 nm is 0.7, to obtain an ABTS analysis solution. 1 mL of CBD with different concentrations and methanol solutions of microcapsules with different concentrations are taken respectively, added with 2 mL of above ABTS analysis solution to the test tube for reacting for 10 min, and then 95% ethanol is used as reference solution to read the absorbance value at 734 nm. The absorbance value of the sample mixed with ABTS analysis solution is marked as A, the absorbance value of the sample mixed with 95% ethanol solution is Ai, and the absorbance value of the single ABTS analysis solution is Aj. The calculation formula is as follows:

$${ABTS}^{+} radical scavenging rate ( \% ) = ( 1 - \frac{A - Ai}{Aj} ) \times 100$$

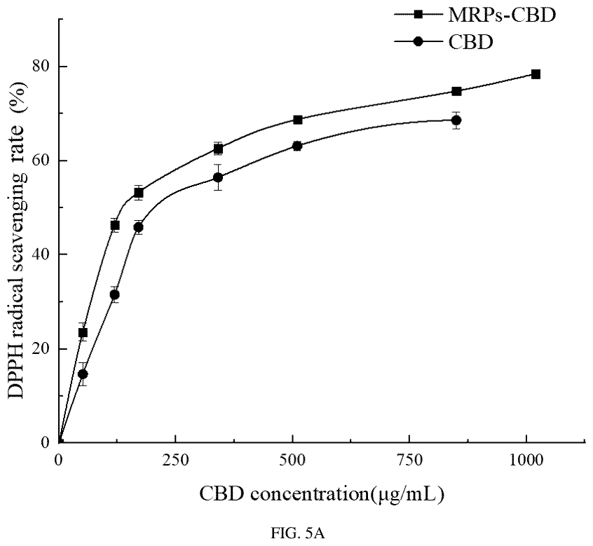

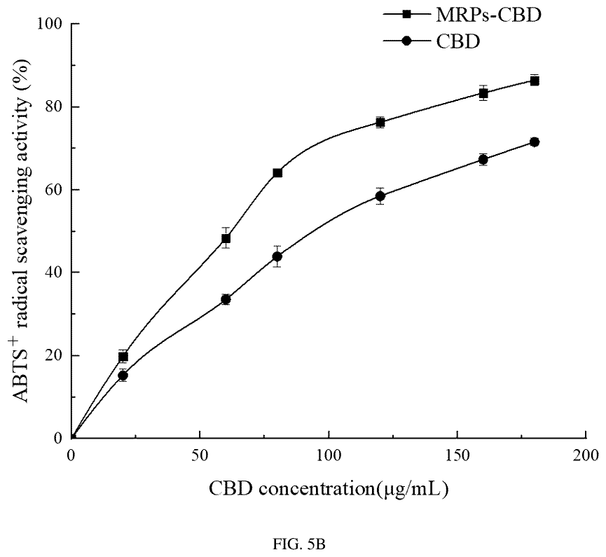

FIG. 5A-FIG. 5B show the radical scavenging rate of MRPs-CBD microcapsules, in which FIG. 5A is results of DPPH radical scavenging rate of the MRPs-CBD microcapsules and FIG. 5B is results of ABTS⁺ radical cation scavenging rate of the MRPs-CBD microcapsules. It can be seen from FIG. 5A-FIG. 5B that the DPPH and ABTS⁺ scavenging rates of unencapsulated CBD and MRPs-CBD microcapsules increase significantly with the increase of CBD concentration (P<0.05). Under the same concentration of CBD in the system, the DPPH and ABTS⁺ scavenging rates of MRPs-CBD microcapsules are higher than those of the unencapsulated CBD. The reason may be that MRPs-CBD microcapsules not only retain the antioxidant capacity of the encapsulated CBD, but also contain the antioxidant activity of the microcapsule wall material MRPs.

In order to verify the stability of the water-soluble cannabidiol microcapsules of the present application, the following tests are conducted:

### I. Determination of Retention Rate of Cannabidiol

Determination of retention rate of CBD in MRPs-CBD microcapsules: the above method to determine CBD content in MRPs-CBD microcapsules is used to accurately determine the initial CBD content in MRPs-CBD microcapsules and the CBD content retained in microcapsules under different treatment methods, and the retention rate of CBD in microcapsules is calculated according to the following formula:

$$retention rate of CBD = \frac{retained CBD content in MRPs - CBD}{initial CBD content in MRPs - CBD} \times 100$$

### II. The Influence of Different Environment on MRPs-CBD Microcapsules

The samples (MRPs-CBD microcapsules) in embodiment 1 are treated according to the following steps.

Influence of temperature on the stability of MRPs-CBD microcapsules

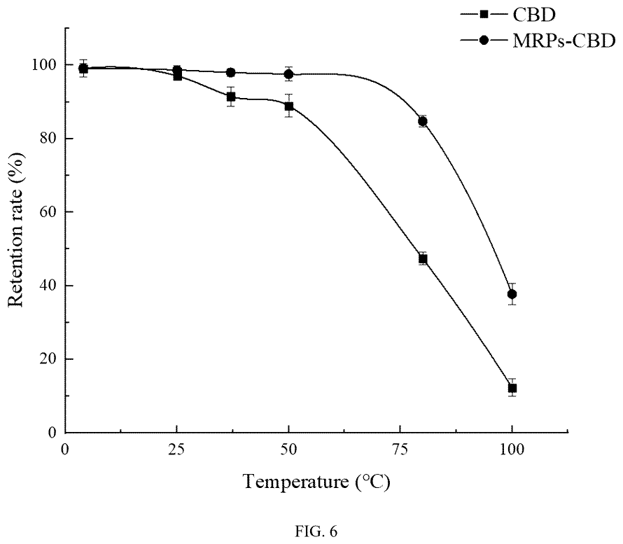

An appropriate amount of samples are weighed, placed in a sealed and dark environment at 4° C., 25° C., 37° C., 50° C., 80° C. and 100° C. for 12 h and taken out, and the retention rate of CBD is determined. See FIG. 6 for the test results.

It can be seen from FIG. 6 that with the increase of temperature, the retention rates of CBD and MRPs-CBD show continuous downward trends. When the temperature is 4° C., there is no significant difference between the retention rate of CBD in MRPs-CBD and the retention rate of non-encapsulate CBD. When the treatment temperature is 25° C., 37° C., 50° C., 80° C. and 100° C., the retention rate of CBD in MRPs-CBD microcapsules is significantly higher than that of non-encapsulate CBD, which shows that microencapsulation may effectively protect CBD and reduce the degradation of CBD in high temperature environment.

### 2. Influence of Environmental pH Value on the Stability of MRPs-CBD Microcapsules

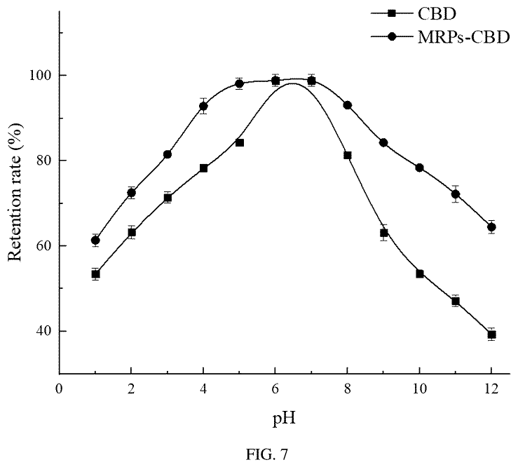

A proper amount of samples are weighed and dissolved in distilled water, and the pH values of the solutions are adjusted as 1, 2, 3, 4, 5, 6, 7, 8, 9, 10, 11 and 12 respectively, and samples are placed in a sealed and dark environment at 4° C. for 12 h, then taken out and its retention rate of CBD is determined. See FIG. 7 for the test results.

It can be seen from FIG. 7 that when the environmental pH value increases from 1 to 6, the retention rates of unencapsulated CBD and MRPs-CBD increase significantly; when the environmental pH value is 6, 7 and 8, there is no significant difference between the retention rates of unencapsulated CBD and MRPs-CBD; when the environmental pH value increases from 8 to 12, the retention rates of unencapsulated CBD and MRPs-CBD decrease significantly. When the environmental pH value is less than 5 or greater than 8, the retention rate of unencapsulated CBD in MRPs-CBD microcapsules is significantly higher than that of unencapsulated CBD. This shows that microcapsules may effectively reduce the loss of encapsulated CBD in strong acid or alkali environment.

### 3. Influence of Oxygen on the Stability of MRPs-CBD Microcapsules

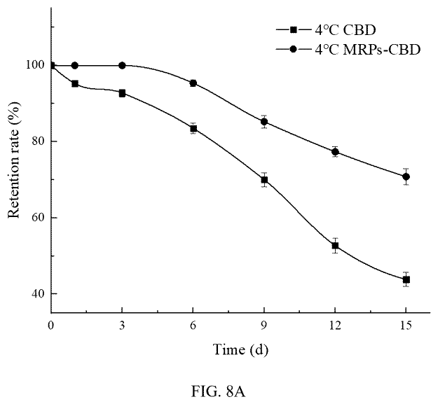

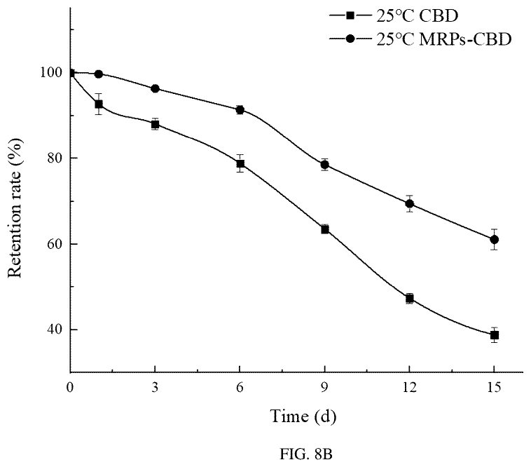

An appropriate amount of samples are weighed, placed at 4° C. and 25° C. for 15 days in the dark and aerobic conditions, and taken out at on 1st, 3rd, 5th, 7th, 9th, 12th and 15th day to determine the retention rate of CBD. See FIG. 8A and FIG. 8B for the test results. FIG. 8A shows an influence of oxygen on the stability of MRPs-CBD microcapsules at 4° C. FIG. 8B shows an influence of oxygen on the stability of MRPs-CBD microcapsules at 25° C.

As can be seen from FIG. 8A and FIG. 8B, with the increase of exposure time to aerobic environment, the retention rate of the unencapsulated CBD and the retention rate of CBD in MRPs-CBD microcapsules decrease significantly. On the 15th day at 4° C. and 25° C., the retention rates of the unencapsulated CBD are only (43.87±1.83) % and (38.85±1.76) %, while the retention rates of CBD in MRPs-CBD microcapsules decrease to (70.77±2.09) % and (61.11±2.38) % respectively. When samples are placed for the same time, under the aerobic environment of 4° C. and 25° C., the stability of MRPs-CBD microcapsules exposed to oxygen is higher than that of the unencapsulated CBD, which may effectively inhibit the CBD from contacting with oxygen.

### 4. Influence of Light on the Stability of MRPs-CBD Microcapsules

### (1) Influence of Incandescent Light on the Stability of MRPs-CBD Microcapsules

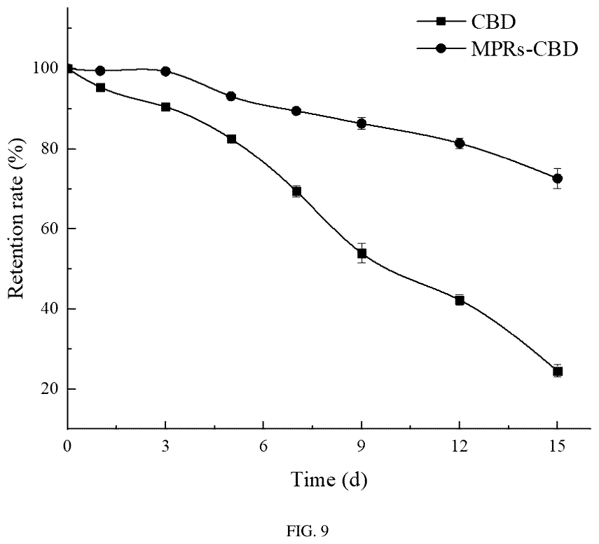

A proper amount of samples are weighed, sealed, placed under incandescent light (25 W) at 4° C. for 15 d, and taken out on the 1st, 3rd, 6th, 9th, 12th and 15th day respectively, and the retention rate of CBD is determined. See FIG. 9 for the test results.

It can be seen from FIG. 9 that the retention rate of CBD in MRPs-CBD microcapsules is significantly higher than that of the unencapsulated CBD after the samples are subjected to the incandescent light. On the 15th day of incandescent light illumination, the retention rate of MRPs-CBD microcapsules decreases to (72.59±2.53) %, and the retention rate of unencapsulated CBD decreases to (24.53±1.56) %. Compared with the unencapsulated CBD, the stability of microencapsulated CBD under incandescent light is obviously improved. Incandescent light causes irreversible isomerization of CBD, and the dense and antioxidant MRPs interface layer in MRPs-CBD microcapsules may weaken the damage of light to CBD.

### (2) Influence of Ultraviolet Irradiation on the Stability of MRPs-CBD Microcapsules

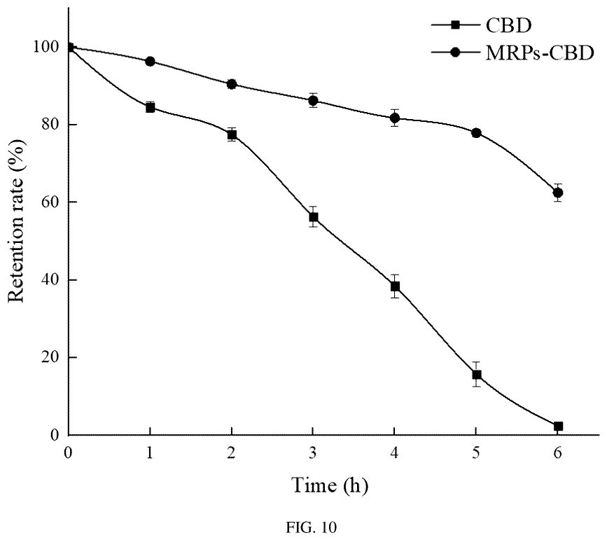

A proper amount of samples are weighed, sealed, put under an ultraviolet lamp (100 W) for 6 h at room temperature, and sampling is conducted every 1 h (the samples are stirred evenly before sampling) to determine the retention rate of CBD. See FIG. 10 for the test results.

As can be seen from FIG. 10, after ultraviolet irradiation, the retention rate of CBD in MRPs-CBD microcapsules is significantly higher than that of unencapsulated CBD. After 6 h illumination, the retention rate of MRPs-CBD microcapsules decreases to (62.49±2.31) %, and the retention rate of unencapsulated CBD decreases to (2.49±0.2.34) %, which is basically photolyzed. Compared with the unencapsulated CBD, the stability of microencapsulated CBD under ultraviolet irradiation is improved.

### III. Study on Release Law of MRPs-CBD Microcapsules

### (1) Release of MRPs-CBD Microcapsules in Simulated Gastric Juice

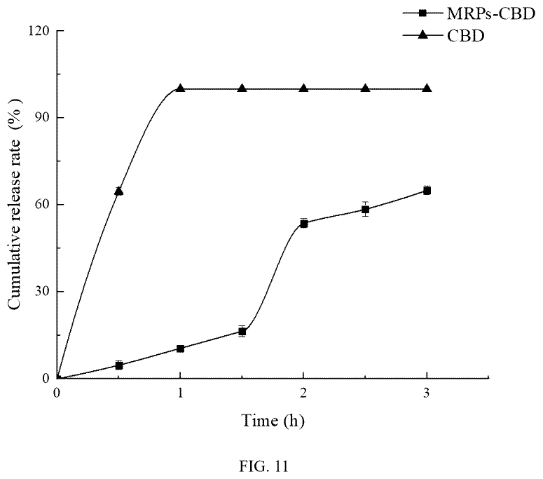

An appropriate amount of samples are weighed and released in simulated gastric juice. Samples are taken according to the time gradient of 0.5, 1.0, 1.5, 2.0, 2.5 and 3.0 h, and the cumulative release rate of CBD is determined. The test results are shown in FIG. 11.

As can be seen from FIG. 11, CBD is completely digested and degraded after being placed in simulated gastric juice for 1 h. With the increase of release time, when the release time increases to 3 h, the cumulative release rate of CBD in MRPs-CBD microcapsules in simulated gastric juice increases significantly, and the release rate is significantly accelerated at 1.5 h, and the cumulative release rate reaches the highest value (64.45±1.46) % at 3 h.

### (2) Release of MRPs-CBD Microcapsules in Simulated Intestinal Juice

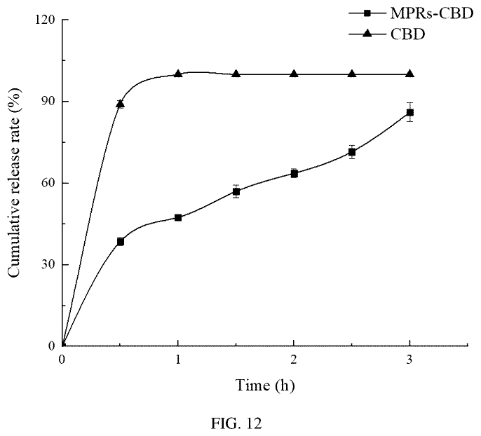

An appropriate amount of samples are weighed and released in simulated intestinal juice. Samples are taken according to the time gradient of 0.5, 1.0, 1.5, 2.0, 2.5 and 3.0 h, and the cumulative release rate of CBD is determined. The test results are shown in FIG. 12.

It can be seen from FIG. 12 that CBD is completely digested and degraded after being placed in simulated intestinal juice for 1 h; when the release time increases to 3 h, the cumulative release rate of CBD in MRPs-CBD microcapsules in simulated intestinal juice increases significantly, and the cumulative release rate reaches the highest value of (86.05±3.45) % at 3 h.

### (3) In-Turn Release of MRPs-CBD Microcapsules in Simulated Gastrointestinal Fluid

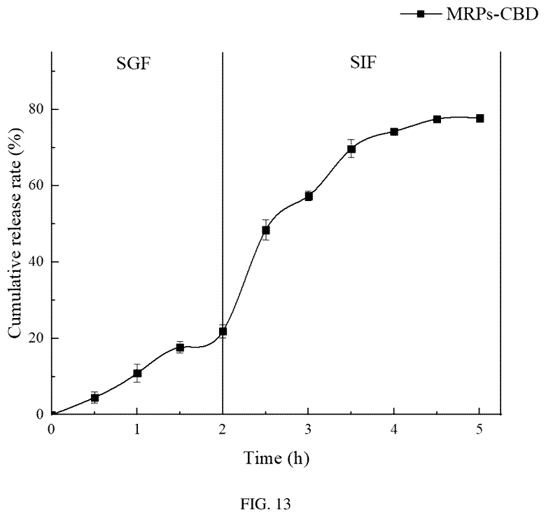

An appropriate amount of samples are weighed and released in simulated gastric juice and intestinal juice in turn. Samples are taken according to the time gradient of 0.5, 1.0, 1.5, 2.0, 2.5, 3.0, 3.5, 4.0, 4.5 and 5.0 h, and the cumulative release rate of CBD is determined. See FIG. 13 for the test results.

It can be seen from FIG. 13 that the cumulative release rate of MRPs-CBD microcapsules increases significantly with the increase of time. When the release time is 2 h, the cumulative release rate increases significantly, indicating that the CBD in the microcapsules is released quickly within 30 min after entering the simulated intestinal juice, probably because trypsin in the simulated intestinal juice has stronger decomposition effect on MRPs-CBD microcapsules. The bioavailability of MRPs-CBD microcapsules is (24.87±2.45) % after in-turn release in simulated gastrointestinal fluid.

### Comparative Embodiment 1

Different from the optimal preparation conditions of Embodiment 1 (oil phase content of 40%, CBD content of 12%, tea saponin content of 0.35%, pH value of external water phase of 6.85), the only difference is that no tea saponin is added.

Results: the encapsulation efficiency and CBD solubility of microcapsules prepared in this comparative embodiment are (53.23±0.27) % and (155.23±0.34) mg/mL, respectively, which are significantly different from the encapsulation efficiency and CBD solubility of microcapsules prepared under optimal preparation conditions in Embodiment 1.

The above-mentioned embodiments only describe the preferred mode of the application, and do not limit the scope of the application. Under the premise of not departing from the design spirit of the application, various modifications and improvements made by ordinary technicians in the field to the technical scheme of the application shall fall within the protection scope defined by the claims of the application.

1. A preparation method of water-soluble cannabidiol microcapsules, comprising following steps:
step 1, adding galactose into an aqueous solution of whey protein isolate, adjusting a pH value to be alkaline, reacting, and cooling to obtain a glycosylation product solution of whey protein isolate-galactose;
step 2, adding an emulsifier into the glycosylation product solution of whey protein isolate-galactose, adjusting a pH value to be acidic or neutral, mixing and stirring with an oil solution of cannabidiol to obtain a crude emulsion, and homogenizing the crude emulsion to obtain a MRPs-CBD emulsion; and
step 3, pre-freezing and vacuum freeze-drying the MRPs-CBD emulsion in turn to obtain the glycosylation product-based water-soluble cannabidiol microcapsules;
wherein in the step 2, the emulsifier is tea saponins; the stirring is specifically emulsifying at 6000−10000 r/min for 2-5 min;
in the step 3, the vacuum freeze-drying is specifically freeze-drying for 18-30 h at −50 to −70° C. and 2-10 Pa;
in the step 2, oil in the oil solution of cannabidiol is sunflower seed oil; a volume ratio of the glycosylation product solution of whey protein-galactose to the oil solution of cannabidiol is 6:4-8:2, the adjusting a pH value is specifically adjusting pH=6.7-6.9.

2. The preparation method according to claim 1, wherein in the step 1, a concentration of the aqueous solution of whey protein isolate is 1-6 wt %; the adjusting a pH value is specifically adjusting pH=8-10; the reacting is specifically reacting at 70-90° C. for 1-5 h.

3. The preparation method according to claim 1, wherein in the step 2, a mass ratio of whey protein to galactose in the glycosylation product solution of whey protein-galactose is 1:1; the emulsifier accounts for 0.05-5% of a mass of the glycosylation product solution of whey protein-galactose.

4. The preparation method according to claim 1, wherein in the step 2, a concentration of the oil solution of cannabidiol is 2-12 wt %.

5. The preparation method according to claim 1, wherein in the step 2, the homogenizing is specifically 4-6 times of cycles at 50-80 MPa.

6. The preparation method according to claim 1, wherein in the step 3, the pre-freezing specifically adopts liquid nitrogen to pre-freeze for 1 min.

7. Water-soluble cannabidiol microcapsules prepared by the preparation method according to claim 1.

8. An application of the water-soluble cannabidiol microcapsules according to claim 7 in preparing anti-oxidation, anti-cancer and anti-inflammatory drugs.
